(19) United States
(12) Patent Application Publication
(10) Pub. No.: US 2007/0292907 A1
(43) Pub. Date: Dec. 20, 2007

(54) Compositions and method for regulating ubiquitin-specific processing proteases

(76) Inventors: Yigong Shi, Plainsboro, NJ (US); Min Hu, Princeton, NJ (US)

Correspondence Address: Pepper Hamilton LLP Firm 21269;50th Floor, One Mellon Center, 500 Grant Street, Pittsburgh, PA, 15219, US

(21) Appl. No.: 11/542,471

(22) Filed: Oct. 3, 2006

Related U.S. Application Data
(60) Provisional application No. 60/723,561, filed on Oct. 3, 2005.

(51) Int. Cl. C07K 7/06; C07K 2/00; C12Q 1/34

(52) U.S. Cl. 435/18; 530/300; 530/329

(57) ABSTRACT

The disclosure contained herein provides compounds and methods for identification of compounds that agonize or antagonize the function of USP14, a member of the ubiquitin-specific processing protease (UBP) family. Over-expression or gain-of-function of UBP's contributes to a range of diseases such as cancer, thus inhibition of USP14 by antagonists may provide a strategy for therapeutic intervention. Loss-of-function or reduced expression of UBP's, or reduced expression of the proteasome, contributes to improper synaptic activity and neurodegenerative diseases, thus activation of USP14 by agonists may provide a strategy for therapeutic intervention.

### CROSS-REFERENCE TO RELATED APPLICATIONS

This application claims the benefit of U.S. Provisional Patent Application Ser. No. 60/723,561, filed on Oct. 3, 2005.

### STATEMENT REGARDING FEDERALLY SPONSORED RESEARCH

Pursuant to 35 U.S.C. §202(c), it is acknowledged that the U.S. Government has certain rights in the embodiments of the disclosure described herein, which was made in part with funds from the National Institutes of Health, Grant No. R01 CA100359 (Princeton University Grant No. 126-6087).

### THE NAMES OF THE PARTIES TO A JOINT RESEARCH AGREEMENT

Not Applicable

### INCORPORATION-BY-REFERENCE OF MATERIAL ON DISC

Not Applicable

### BACKGROUND

The disclosure contained herein generally relates to novel methods for the identification of compounds that agonize or antagonize the function of ubiquitin-specific processing proteases (UBP). More specifically, the disclosure provides for peptides and peptidomimetics capable of activating or inhibiting USP14, and thus modulating USP14's activity in a range of disease, such as cancer and those associated with improper synaptic activity in mammals. The disclosure further provides methods useful for the screening of agonists or antagonists of USP14 and using these compounds for therapeutic purposes and rational drug design.

Protein ubiquitination plays an essential role in the regulation of many cellular processes in eukaryotes (Glickman and Ciechanover (2002) Physiol Rev 82:373-428; Hershko et al. (2000) Nat Med 6:1073-1081; Pickart (2004) Cell 116:181-190). Ubiquitin is a highly conserved 76 amino acid polypeptide. Through sequential action of three classes of enzymes known as ubiquitin-activating enzyme (E1), ubiquitin-conjugating enzyme (E2) and ubiquitin ligase (E3), ubiquitin is linked to target proteins by an isopeptide bond between the C-terminal carboxylate group of ubiquitin and the lysine E-amino group of the acceptor protein. Ubiquitination is tightly regulated, and aberrations in this pathway are known to lead to a variety of clinical disorders (Chung et al. (2001) Trends Neurosci 24:S7-S14; Schwartz and Ciechanover (1999) Annu Rev Med 50:57-74). A major function of ubiquitination is to target proteins for degradation by the 26S proteasome.

Protein deubiquitination has been identified as an important regulatory step in the ubiquitin-dependent pathways (Amerik and Hochstrasser (2004) Biochim Biophys Acta 1695:189-207; D'Andrea and Pellman (1998) Crit. Rev Biochem Mol Biol, 33:337-352; Kim et al. (2003) J Biochem (Tokyo) 134:9-18; Wilkinson et al. (2000) J Biol Chem 275:15182-15192; Wing (2003) Int J Biochem Cell Biol 35:590-605). Deubiquitination is carried out by the deubiquitinating enzymes (DUBs), which catalyze the hydrolysis of the isopeptide bond in ubiquitin-protein conjugates. There are at least five conserved families of DUBs, of which the ubiquitin-specific processing proteases (UBP's) are the largest, with more than 60 members identified in the human genome.

The 26S proteasome is a multi-subunit machine that degrades polyubiquitinated protein substrates (Pickart and Cohen (2004) Nat Rev Mol Cell Biol 5:177-187; Zwickl et al. (1999) Philos Trans R Soc Lond B Biol Sci 354:1501-1511). A number of auxiliary proteins, such as ubiquitinating enzymes (Verma et al. (2000) Mol Biol Cell 11:3425-3439; Xie and Varshavsky (2000) Proc Natl Acad Sci USA 97:2497-2502) and deubiquitinating enzymes (Borodovsky et al. (2001) Embo J 20:5187-5196; Guterman and Glickman (2004b) Curr Protein Pept Sci 5:201-211; Lam et al. (1997) Nature 385:737-740; Leggett et al. (2002) Mol Cell 10:495-507; Papa et al. (1999) Mol Biol Cell 10:741-756; Verma et al. (2000) supra), specifically associate with the proteasome. The proteasome-associated deubiquitinating enzymes include UCH37 (Lam et al. (1997) supra), a member of the Ubiquitin C-terminal hydrolases (UCHs) family of small ubiquitin-specific proteases, POH1/Rpn11, a proteasome 19S cap subunit belonging to the Jab1/MPN domain-associated metalloisopeptidase (JAMM) family that lacks the standard cysteine protease signature (Verma et al. (2002) Science 298:611-615; Yao and Cohen (2002) Nature 419:403-407), and USP14/Ubp6 (Borodovsky et al. (2001) supra; Chernova et al. (2003) J Biol Chem 278:52102-52115; Leggett et al. (2002) supra), a member of the UBP family. These deubiquitinating enzymes help to remove the polyubiquitin moiety from protein substrates before or during translocation into the catalytic chamber of the proteasome for degradation. While the editing function of deubiquitinating enzymes can rescue poorly ubiquitinated protein substrates from degradation by the 26S proteasome (Lam et al. (1997) supra), it is the ubiquitin-recycling function that is critical for maintaining the free ubiquitin pool in cells and which protects the proteasome from being jammed by the ubiquitin chains attached to substrates (Chernova et al. (2003) supra; Guterman and Glickman (2004a) supra; Hanna et al. (2003) Mol Cell Biol 23:9251-9261; Leggett et al. (2002) supra; Verma et al. (2002) supra; Yao and Cohen (2002) supra). Both USP14 and Ubp6 contain a ubiquitin-like (Ubl) domain at the N-terminus (Wyndham et al. (1999) Protein Sci 8:1268-1275); the Ubl of Ubp6 has been shown to be responsible for the association with 26S proteasomes. This association results in the dramatic enhancement of Ubp6 deubiquitinating activity in vitro, although the underlying mechanism remains unclear (Leggett et al. (2002) supra).

Mutations that eliminate Rpn11 deubiquitinating activity are lethal for yeast and lead to the accumulation of ubiquitinated degradation substrates (Verma et al. (2002) supra; Yao and Cohen (2002) supra). Lesions in the proteasome-associated UBP enzyme, USP14/Ubp6, have pronounced, but milder effects. In mice, defective USP14 results in abnormal synaptic transmission and ataxia (Wilson et al. (2002) Nat Genet 32:420-425). In budding yeast, deletion of UBP6 severely impairs growth under various conditions of stress and causes a major depletion of the cellular ubiquitin pool; indeed, ubiquitin overexpression can suppress the phenotypes of ubp6Δ yeast (Chernova et al. (2003) supra; Hanna et al. (2003) supra). Although both POH1/Rpn11 and USP14/Ubp6 function to recycle ubiquitin, the more drastic effects of mutations in POH1/Rpn11 suggest that it bears the greater responsibility for releasing ubiquitin from proteasomal substrates. Nonetheless, rpn11 and ubp6 mutations are synthetically lethal in yeast (Guterman and Glickman (2004a) supra), which suggests that these structurally distinct deubiquitinating enzymes have overlapping functions.

Increased expression of USP14 has been linked to colorectal cancer in human patients (Shinji et al. (2006) Oncol Rep 15:539-543). Immunohistochemically, USP14 was absent or weakly localized in the cytoplasm of normal colorectal epithelial cells, but was strongly detected in the cytoplasm of cancer cells. USP14 expression correlated with pathological stage, and lymph node and liver metastases, where the overall survival rate was less in patients with a high USP14 expression level than in those with a low USP14 expression level.

Understanding how these deubiquitinating enzymes are regulated and how their substrates are identified are major unsolved problems in the area of ubiquitin-dependent degradation.

The UBP's are cysteine proteases that contain highly divergent sequences and exhibit strong homology mainly in two regions that surround the catalytic Cys and His residues; these are the so-called Cys Box (˜19 amino acids) and the His Box (60-90 amino acids) (D'Andrea and Pellman (1998) supra; Papa and Hochstrasser (1993) Nature 366:313-319). The structure of the catalytic core domain of HAUSP or “herpes-associated ubiquitin-specific protease” (also known as USP7), revealed a tripartite architecture comprising Fingers, Palm, and Thumb domains (Hu et al. (2002) supra). Given the highly divergent sequences and scarce structural information, it is unclear whether this three-domain architecture is generally conserved among other UBP's. In addition, the catalytic residues in HAUSP are mis-aligned prior to substrate binding. Binding by ubiquitin aldehyde induces a drastic conformational change in the active site that realigns the catalytic triad residues for catalysis (Hu et al. (2002) supra). It is unknown whether this is a general activation mechanism among UBP's.

The present lack of structural information for the aforementioned UBP's prevents their use as therapeutic, targets for drug screening and rational drug design. Thus, a need exists to identify the structural features and regulatory mechanisms of these proteins that underlie their ability to facilitate deubiquitination of their specific substrates.

### SUMMARY

An embodiment contained herein relates to a novel method for the identification of compounds that agonize or antagonize the function of ubiquitin-specific processing proteases (UBP's). More specifically, the disclosure provides for peptides and peptidomimetics capable of activating or inhibiting USP14, and thus modulating USP14's activity. Such compounds may be useful as therapeutics in a range of diseases such as cancer and those associated with improper synaptic activity. The disclosure further provides methods useful for the screening of agonists or antagonists of USP14 and using these agonists or antagonists for therapeutic purposes and rational drug design.

The UBP family of deubiquitinating enzymes plays an essential role in numerous cellular processes. Mammalian USP14 (Ubp6 in yeast) is unique among known UBP enzymes in that it is activated catalytically upon specific association with the 19S recognition particle (19S RP) of the 26S proteasome. Here we report the crystal structures of the 45-kDa catalytic domain of USP14 in isolation and in a complex with ubiquitin aldehyde, which reveal distinct structural features. In the absence of ubiquitin binding, the catalytic cleft leading to the active site of USP14 is blocked by two surface loops. Binding by ubiquitin shows that a significant conformational change which translocates the two surface loops occurs, thus allowing the ubiquitin C-terminus access to the active site. These structural observations, in conjunction with biochemical characterization, identify important regulatory mechanisms for USP14.

Thus, in an embodiment of the disclosure, the USP14 binding peptides or peptidomimetics identified may be utilized in various assays to screen for and identify compounds capable of acting as agonists and antagonists of the USP14 catalytic activity. Antagonists may be useful as anti-cancer drugs for the treatment of the aberrant cell proliferation observed in cancer. Agonists may be useful in regulating synaptic activity in mammals for the treatment of neurodegenerative diseases.

In another embodiment, the USP14 binding peptides or peptidomimetics may be used in a cell free binding assay to screen for USP14-binding antagonists and agonists. Such assays may be used for high throughput screening for candidate drugs, e.g. in a chemical library or produced by rational drug design (mimetics) as disclosed herein.

In yet another embodiment of the instant disclosure, small molecules or peptidomimetics or mimetics of the antagonists or agonists of USP14 activity may be used as a therapeutic agent for the treatment of diseases involving USP14, including but not limited to, cancer and neurodegenerative diseases. Accordingly, an embodiment of the disclosure comprises a therapeutic composition of a small molecule, peptidomimetic or mimetic of the antagonists or agonists of USP14 activity and a pharmaceutically acceptable carrier. A further embodiment of the disclosure comprises administering to a cell a therapeutically effective amount of the therapeutic composition containing the small molecule, peptidomimetic or mimetic of the antagonists or agonists of USP14 activity to stimulate or inhibit the activity of USP14. Stimulating the USP14 activity may be useful for the treatment of neurodegenerative diseases, while inhibiting the activity may be useful for the treatment of cancer. In another embodiment, the cell is contained within a tissue, and the tissue is preferably located in a living organism, preferably an animal, more preferably a mammal, and most preferably a human.

### BRIEF DESCRIPTION OF SEVERAL VIEWS OF THE DRAWINGS

The file of this patent contains at least one drawing/photograph executed in color. Copies of this patent with color drawing(s)/photograph(s) will be provided by the Office upon request and payment of the necessary fee. All figures where structural representations are shown were prepared using MOLSCRIPT (Kraulis (1991) J Appl Crystallogr 24:946-950) and GRASP (Nicholls et al. (1991) Proteins: Struct Funct Genet. 11:281-296).

Aspects, features, benefits and advantages of the embodiments herein will be apparent with regard to the following description, appended claims, and accompanying drawings, where:

FIG. 1A is a schematic depiction of the overall structure of the 45-kDa catalytic domain of USP14 (residues 91-494). The structure comprises three domains: Fingers (in green), Palm (in blue), and Thumb (in gold). The active site, comprising the Cys Box (in cyan) and the His Box (in magenta), is located between the Palm and the Thumb. The predicted ubiquitin-binding site is indicated by a black oval circle. The surface representation is shown on the right. Note the absence of the binding groove for the C-terminus of ubiquitin.

FIG. 1B is a schematic diagram showing a comparison of the structures of the catalytic domain between USP14 and 1-HAUSP in a stereo view. USP14 and HAUSP are shown in blue and white, respectively. The active site of free USP14 is covered by two cross-over loops BL1 and BL2 (in red). The catalytic Cys in USP14 and HAUSP are highlighted in yellow.

FIG. 1C is a schematic diagram showing a comparison of the structures of the catalytic domain between USP14 and Ubp6 in a stereo view. Note that two surface loops (green) in Ubp6 adopt very similar positions as the BL1 and BL2 loops (red) in USP14.

FIG. 2 is a schematic depiction of a sequence alignment of USP14 with its yeast homolog Ubp6 and human HAUSP. Conserved residues are shaded in yellow whereas the catalytic triad residues are highlighted in red. The secondary structural elements above the sequences are indicated for free USP14 (lower) and HAUSP (upper). The four black arrow heads indicate the positions where Cys residues are supposed to be located in a zinc ribbon (Krishna and Grishin (2004) Cell Cycle 3:1046-1049). The coloring scheme for the secondary structural elements of free USP14 is the same as in FIG. 1. Sequence alignment employed the programs ClustalW. Entries shown are from the SwissProt Database: HAUSP (Human; SW:Q93009); USP14 (Human; SW:P54578); UBP6 (S. cerevisiae; SW:P35127).

FIG. 3A is a schematic diagram of the structure of the active site of USP14. The 2Fo-Fc electron density at the active site region contoured at 1.8σ. The Cys and His boxes are colored cyan and magenta, respectively.

FIG. 3B is a schematic diagram of the structure of the catalytic triad residues of USP14 when poised for catalysis. Shown here is a stereo comparison of the active sites of USP14 and HAUSP. The coloring scheme for USP14 is the same as in FIG. 1. HAUSP is shown in green. Catalytic triad residues and the oxyanion-coordinating residue are shown. Hydrogen bonds are represented by red dashed lines.

FIG. 3C is a schematic diagram of the structure of the binding cleft of the C-terminus of ubiquitin when blocked by two surface loops in USP14. The binding region for the C-terminus of ubiquitin is shown in two surface representations: solid (left panel) and transparent (right panel). The C-terminus of ubiquitin (green) is placed after superposition of the HAUSP-Ubal structure onto USP14. Several residues of USP14, including Phe331, Tyr333 and Ser432, sterically clash with the C-terminus of ubiquitin.

FIG. 3D is a schematic diagram comparing the structure of the active site conformations in USP14 and Ubp6. Residues from USP14 are labeled whereas the corresponding residues from Ubp6 are shown in parentheses. The coloring scheme for USP14 is the same as in FIG. 1. Ubp6 is shown in gray. Catalytic triad residues and the oxyanion-coordinating residue are shown. Hydrogen bonds are represented by red dashed lines.

FIG. 4A is a schematic diagram of the structure of the USP14-Ubal complex. Overall structure of the catalytic core domain of USP14 (91-494, blue) covalently bound to Ubal (in green). The Cys and His Boxes are colored cyan and magenta, respectively. The catalytic Cys 114 is shown in ball-and-stick.

FIG. 4B is a schematic diagram showing the large conformational change near the active site induced by Ubal binding. The ubiquitin C-terminus-binding region of USP14 in isolation (in orange) and in complex with Ubal (in blue) are superimposed and shown in stereo. The C-terminal tail of Ubal is shown in green. Note the conformational changes on the two surface loops, which allow the opening of the binding cleft for the C-terminus of ubiquitin. Amino acids are shown in ball-and-stick.

FIG. 4C is a schematic comparison of the conformation of the blocking loops in USP14 and in HAUSP. Two conserved residues from USP14, Phe331 and Tyr333 (Phe409 and Tyr411 in HAUSP), make van der Waals interactions with residues in Ubal. The BL1/BL2 loops and Ubal in the USP14-Ubal complex are colored blue and green, respectively. The HAUSP-Ubal complex is colored gray. The side chains from the USP14/HAUSP and Ubal are shown in yellow and orange, respectively.

FIG. 5A is a depiction of the fluorescence intensity as detected by a cooled CCD camera from an SDS polyacrylaminde gel showing polyubiquitin chain disassembly by USP14. USP14 interacts with the 19S regulatory particle (19S RP) of the 26S proteasome and is activated upon binding. The proximal end ubiquitin of the triUb chain is fluorescently labeled. As indicated by the sequential appearance of fluorescent bands corresponding to labeled diUb and monoUb, USP14 preferentially cleaves ubiquitin from the distal end of the triUb chain.

FIG. 5B is a depiction of USP14 binding to the 19S RP of the 26S proteasome, as detected by an immunoblot of an SDS polyacrylamide gel. An approximately equi-molar amount of GST-USP14, GST-USP14 (91-494), GST-UBL, or GST (control) was used for each experiment. An equal amount of 26S proteasome or the 19S complex (PA700) was used within the same set of experiments. Only full-length (GST-USP14 (FL)) or the isolated Ubl domain (GST-UBL), but not Ubl-deleted USP14 (GST-Core domain) exhibited binding in GST pull-down assays. Anti-SI antibody was used to detect 26S proteasome or 19S RP complexes that were bound and then eluted with glutathione.

FIG. 5C is a depiction of USP14 reactivity with UbVS in the presence of the 19S RP of the 26S proteasome, as detected by an immunoblot of an SDS polyacrylamide gel.

### DETAILED DESCRIPTION

Before the present compositions and methods are described, it is to be understood that they are not limited to the particular compositions, methodologies or protocols described, as these may vary. It is also to be understood that the terminology used in the description is for the purpose of describing the particular versions or embodiments only, and is not intended to limit their scope in the present disclosure which will be limited only by the appended claims. Various scientific articles, patents and other publications are referred to throughout the specification. Each of these publications is incorporated by reference herein in its entirety.

It must also be noted that as used herein and in the appended claims, the singular forms “a”, “an”, and “the” include the plural reference unless the context clearly dictates otherwise. Thus, for example, reference to an “antagonist” is a reference to one or more antagonists and equivalents thereof known to those skilled in the art, and so forth. Unless defined otherwise, all technical and scientific terms used herein have the same meanings as commonly understood by one of ordinary skill in the art. Although any methods and materials similar or equivalent to those described herein can be used in the practice or testing of embodiments disclosed, the preferred methods, devices, and materials are now described.

“Optional” or “optionally” means that the subsequently described event or circumstance may or may not occur, and that the description includes instances where the event occurs and instances where it does not. In addition, the word “comprising” as used herein means “including, but not limited to”. Throughout the specification of the application, various terms are used such as “primary”, “secondary”, “first”, “second”, and the like. These terms are words of convenience in order to distinguish between different elements, and such terms are not intended to be limiting as to how the different elements may be utilized.

As used herein, “isolated” means altered or removed from the natural state through human intervention. For example, a USP14 naturally present in a living animal is not “isolated,” but a synthetic USP14, or a USP14 partially or completely separated from the coexisting materials of its natural state is “isolated.” An isolated USP14 can exist in substantially purified form, or can exist in a non-native environment such as, for example, a cell into which the USP14 has been delivered.

The terms “mimetic,” “peptide mimetic” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinantly or chemically modified peptides, as well as non-peptide agents such as small molecule drug mimetics, as further described below.

By “pharmaceutically acceptable”, it is meant the carrier, diluent or excipient must be compatible with the other ingredients of the formulation and not deleterious to the recipient thereof. As used herein, the term “pharmaceutically acceptable salts, esters, amides, and prodrugs” refers to those carboxylate salts, amino acid addition salts, esters, amides, and prodrugs of the compounds of the present disclosure which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention.

The terms “therapeutically effective” or “effective”, as used herein, may be used interchangeably and refer to an amount of a therapeutic composition of embodiments of the present invention (e.g. one or more of the peptides or mimetics thereof). For example, a therapeutically effective amount of a composition comprising a mimetic is a predetermined amount calculated to achieve the desired effect. As used herein, an “effective amount” of the antagonist or mimetic is an amount sufficient to cause antagonist mediated inhibition of USP14, and thus modulate USP14's activity in a range of diseases, such as cancer. As used herein, the term “cancer” refers to any type of cancer, including, but not limited to, ovarian cancer, leukemia, lung cancer, colon cancer, CNS cancer, melanoma, renal cancer, prostate cancer, breast cancer, and the like.

In accordance with the present disclosure, the specific structural bases for the recognition and binding of substrates to USP14 have been elucidated. The X-ray crystallography of the USP14 catalytic domain reported herein suggests that the association of USP14 with the 26S proteasome may promote a conformational change in a loop structure that occludes the active site for deubiquitination in free USP14. Further, the high resolution crystal structure of the catalytic domain of USP14 in the presence and absence of the substrate ubiquitin aldehyde (Ubal) reveals the specific residues involved in recognition and catalysis. Thus, embodiments of the present disclosure address the need for structural information on ubiquitin-specific processing proteases (UBP), and indicate novel methods for the identification of compounds that antagonize or agonize the function of these UBP's. More specifically, the disclosure provides for peptides and peptidomimetics capable of inhibiting or enhancing USP14 activity, and thus modulating USP14's activity in a range of diseases, such as cancer or neurodegenerative diseases.

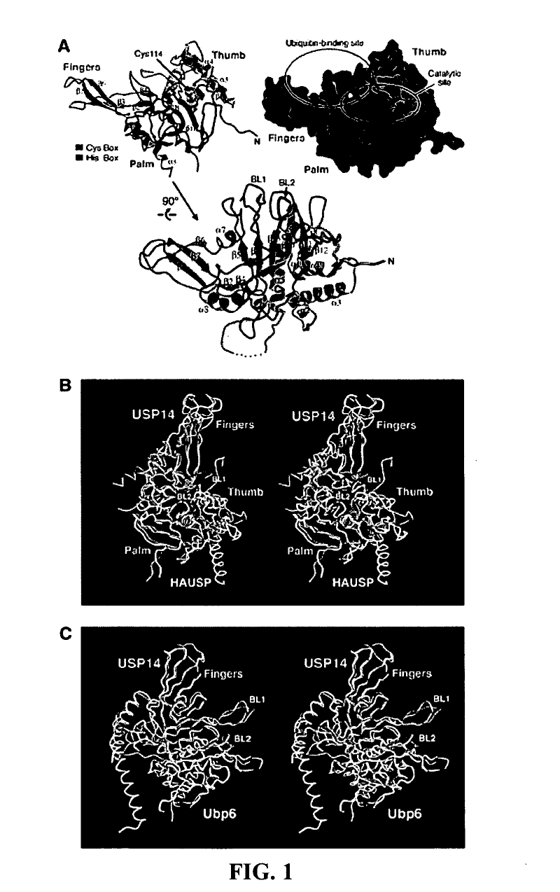

The full-length human USP14 contains 494 amino acids, with a 9-kDa Ubl domain at its N-terminus followed by a 45-kDa catalytic domain. To investigate the function and catalytic mechanism of USP14, we crystallized and determined the structure of the USP14 catalytic domain (residues 91-494) at 3.2 Å resolution using multi-wavelength anomalous dispersion (Table 1 and FIG. 1A). In the crystals, there are three molecules of USP14 per asymmetric unit, which have a pair-wise root-mean-square deviation (RMSD) of approximately 1 Å. These three molecules exhibit identical structural features important for this discussion. Hence, for simplicity, we limit our discussion to one such molecule. The USP14 coordinates are deposited with the protein data bank and are given the accession code 1AYN. The USP14-Ubal complex coordinates are deposited with the protein data bank and are given the accession code 1AYO.

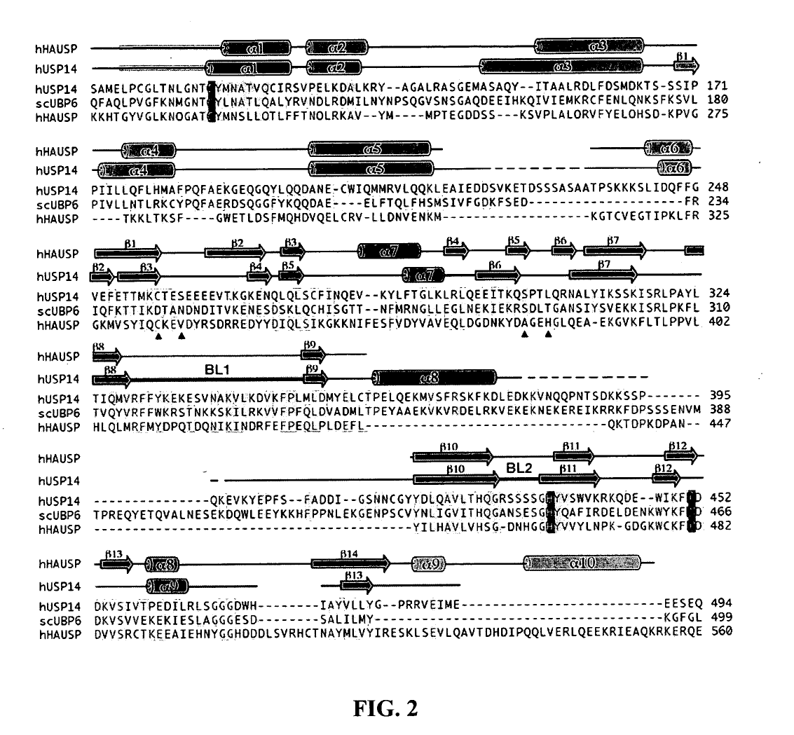

Similar to HAUSP (Hu et al. (2002) supra), the catalytic domain of USP14 resembles an extended right hand comprised of three domains: Fingers, Palm, and Thumb (FIGS. 1 and 2). The three-domain organization creates a prominent binding surface between the Fingers and the Palm-Thumb scaffold, which is predicted to bind to ubiquitin. The Thumb contains 6 α helices (α1-α6) and one short β strand (β1), with the N-terminal Cys Box adopting an extended conformation. The Palm consists of a 6-stranded (β5, β8, β10-β13) central P sheet, three α helices (β7-β9), one short β strand (β9), and several surface loops. Notably, two surface loops hover above and partially fill the predicted binding pocket for the C-terminus of ubiquitin (FIG. 1A). These two loops are named blocking loops 1 and 2 (BL1 & BL2, FIGS. 1B and 2). The Fingers comprise five β strands (β2-β4, β6, and β7). In contrast to the HAUSP structure, packing interactions in the USP14 catalytic domain between the central β sheet in the Palm and the globular Thumb do not give rise to an inter-domain cleft between the Palm and the Thumb (FIG. 1A, right panel), which appears to be needed for accommodation of the ubiquitin C terminus.

The structure of the USP14 catalytic domain resembles that of the HAUSP catalytic core domain, with an RMSD of 1.7 Å for 238 aligned backbone Cα atoms (FIG. 1B). Given only 13.9 percent sequence identity between USP14 and HAUSP (FIG. 2), the preservation of the three-domain architecture suggests that this organization may be generally conserved among all members of the UBP family of proteins. In support of this conclusion, all residues that contribute to the structural integrity of the Fingers, Palm, and the Thumb are highly conserved among HAUSP, USP14, Ubp6, and other representative UBP's (FIG. 2).

Despite overall structural similarity, USP14 and HAUSP exhibit a number of significant local structural differences (FIGS. 1B and 2). Compared to HAUSP, USP14 contains one additional α helix (α8) in the Palm domain and a few extended surface loops in the Thumb and Palm domains, but is missing two C-terminal helices. In addition, a short β strand (β113) in the His Box of the HAUSP core domain structure is replaced by a surface loop in USP14. There are also apparent local structural differences in the Fingers domain. Two extended strands β1 and β2 in the HAUSP structure are replaced by a pair of short β strands (β2 & β3) followed by a surface loop and a short strand β4 in USP14. Moreover, two short βstrands (β4 & β5) in HAUSP are reduced to a loop conformation in USP14.

Ubp6 is the functional homologue of USP14 in Saccharomyces cerevisiae and shares 31 percent sequence identity with USP14 in the catalytic core domain. Thus, it is not surprising that the structure of the USP14 catalytic domain is also similar to that of the catalytic core domain from Ubp6 (protein data bank accession code 1VJV), with an RMSD of 1.2 Å for 288 aligned backbone Cα atoms. Interestingly, similar to USP14, Ubp6 contains two surface loops that are located above and partially block the predicted binding pocket for the C-terminus of ubiquitin (FIG. 1C). These two surface loops exhibit nearly identical topology as BL1 and BL2 in USP14 (FIG. 1C). In contrast to the USP14-HAUSP comparison, the local structural differences between USP14 and Ubp6 concentrate in surface regions while the core structural elements are nearly identical to each other (FIG. 1C).

TABLE 1
Summary of crystallographic analysis.

| | Data sets | | | | |
|---|---|---|---|---|---|
| | Native | Native | Peak | Inflection | Remote |
| | (USP14) | (USP14-Ubal) | (Se1) | (Se2) | (Se3) |
| Wavelength (Å) | 1.10 | 0.976 | 0.9793 | 0.9795 | 0.9500 |
| Space group | P212121 | P3121 | P212121 | P212121 | P212121 |
| Resolution (Å) | 99-3.2 | 99-3.5 | 99-3.35 | 99-3.35 | 99-3.35 |
| Unique reflections | 27,320 | 11,099 | 24,378 | 24,012 | 24,805 |
| Completeness | 98.9% (96.3%) | 98.0% (92.8%) | 99.7% (98.6%) | 99.8% (99.4%) | 99.7% (99.1%) |
| (outer shell) | | | | | |
| R_{sym} | 0.078 (0.53) | 0.117 (0.54) | 0.143 (0.72) | 0.123 (0.67) | 0.132 (0.77) |
| (outer shell)¹ | | | | | |
| Data redundancy | 5.6 | 3.3 | 9.6 | 7.2 | 7.2 |
| Average 1/σ | 26.4 (2.8) | 10.9 (2.0) | 19.5 (2.8) | 17.8 (2.6) | 16.6 (2.2) |
| (outer shell) | | | | | |
| Anomalous difference (%) | | | 11.9 | 10.0 | 10.6 |
| Cullis R factor | | | 0.58 | 0.60 | 0.66 |
| Phasing power | | | 2.56/1.84 | 2.36/1.75 | 1.79/1.29 |
| (centric/acentric) | | | | | |
| Mean figure of Merit | | | 0.48 | | |
| (20-3.35 Å) | | | | | |

Refinement statistics:

| | | |
|---|---|---|
| Total | Completeness | RMSD³ |

| Resolution | Number of | number | of data | R-factor² | Bond | Angle |
|---|---|---|---|---|---|---|
| Range (Å) | Reflections | of atoms | (Outer shell) | (R-free) | (Å) | (deg.) |
| 99-3.2 | 25,614 | 8,166 | 90.1% | 0.261 | 0.011 | 1.56 |
| (USP14) | (\|F\| > σ) | | (83.4%) | (0.322) | | |
| 99-3.5 | 11,095 | 3,412 | 97.9% | 0.29 | 0.013 | 1.91 |
| (USP14-Ubal) | (\|F\| > σ) | | (96.1%) | (0.33) | | |

¹R_{sym} = ΣₕΣᵢ | I_{h, i} − Iₕ |/ΣₕΣᵢ | I_{h, l}, where Iₕ is the mean intensity of the i observations of symmetry related reflections of h.
²R = Σ | F_{obs} − F_{calc} |/ΣF_{obs}, where F_{obs} = Fₚ, and F_{calc} is the calculated protein structure factor from the atomic model (R_{free} was calculated with 5% of the reflections).
³RMSD (root-mean-square deviation) in bond lengths and angles are the deviations from ideal values.

Active Site Conformation

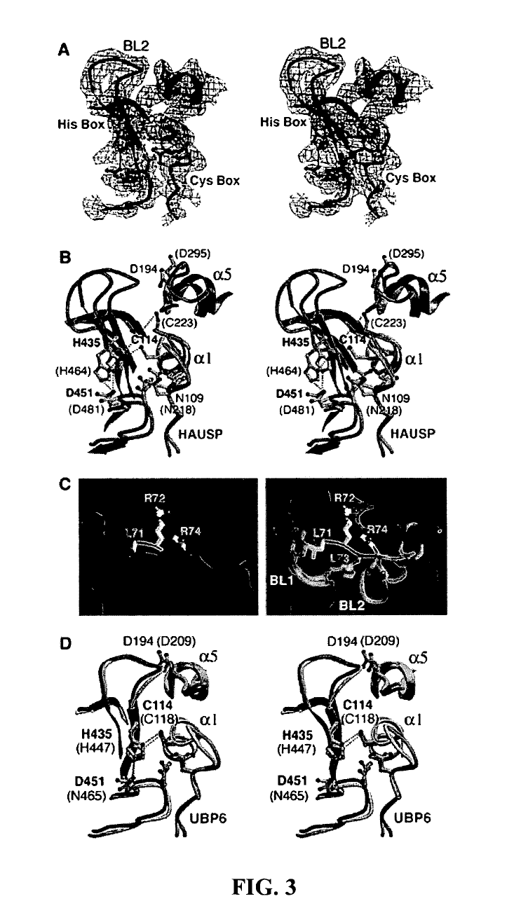

One feature revealed by the structure of the isolated HAUSP catalytic core domain is the mis-aligned active site. In the free HAUSP structure, the catalytic histidine (His464) is nearly 10 Å away from the catalytic cysteine (Cys223), which may be out of range for any meaningful interaction. In contrast to the deformed active site conformation of HAUSP, the active site of free USP14 is already well formed prior to substrate binding (FIG. 3A). Superposition of the active sites between HAUSP and USP14 revealed the differences (FIG. 3B). The N_{δ1} atom in the imidazole ring of the candidate catalytic histidine (His435) is approximately 3.3 Å away from the S_{γ} atom in the side chain of the catalytic cysteine (Cys114), consistent with a hydrogen bond distance. A third residue, Asp451, stabilizes His435 by accepting a hydrogen bond from its N_{ε1} atom. Thus Cys114, His435, and Asp451 form a catalytic triad in the active site of free USP14, and the catalytic mechanism of USP14 appears to parallel that of the papain family of cysteine proteases.

The fact that the catalytic triad residues in free USP14 exist in a productive conformation suggests that free USP14 is already poised for catalysis. This conclusion is in contrast to the observation that free USP14 exhibits only a low level of deubiquitinating activity toward substrates and weak reactivity with ubiquitin vinylsulfone (UbVS, see below). An examination of the USP14 structure reveals a plausible explanation. While not wishing to be bound by theory, the catalytic triad residues may have already adopted a productive conformation. Access to these residues by ubiquitin may be restricted, however (FIG. 3C). Right above the active site of USP14, the two surface loops BL2 and BL1 are positioned close to the predicted binding groove for the C-terminus of ubiquitin. Superposition of USP14 with the HAUSP-Ubal complex revealed that loops BL2 and BL1 would likely block access of the C-terminus of ubiquitin to the active site of USP14. Thus, the blockade of the ubiquitin C-terminus binding groove by loops BL2 and BL1 appears to be required to be removed in order for USP14 to catalyze deubiquitination.

Consistent with this analysis, the active site of the yeast Ubp6 protein (protein data bank accession code 1VJV) adopts a highly similar conformation to that of USP14 (FIG. 3D). The catalytic triad residues in Ubp6, Cys118, His447, and Asn465, can be superimposed with those from USP14 with a RMSD of 0.1 Å. The N_{δ1} atom in the imidazole ring of His447 is within hydrogen bond distance of the side chain of Cys118, and this interaction is buttressed by a second hydrogen bond from Asn465 to His447 (FIG. 3D). Thus the catalytic triad residues Cys118, His447, and Asn465 in Ubp6 already exist in a productive conformation. Similar to USP14, this observation suggests that the blockade of the ubiquitin C-terminus binding groove by two surface loops should be removed before Ubp6 can catalyze deubiquitination.

Overall Structure of USP14-Ubal Complex

Ubiquitin aldehyde (Ubal) is an ubiquitin derivative in which the C-terminal carboxylate is replaced by an aldehyde. Ubal is a potent covalent inhibitor of most DUBs as it forms a thiohemiacetal with the catalytic cysteine, mimicking a reaction intermediate (Hershko and Rose (1987) Proc Natl Acad Sci USA 84:1829-1833; Johnston et al. (1999) Embo J 18:3877-3887; Pickart and Rose (1985) J Biol Chem 260:7903-7910). To further elucidate the catalytic mechanism of USP14, we prepared Ubal and reconstituted a covalent complex between USP14 and the inhibitor. We crystallized this binary complex and determined its structure at 3.5 Å resolution by molecular replacement (Table 1). There is one USP14-Ubal complex in each asymmetric unit.

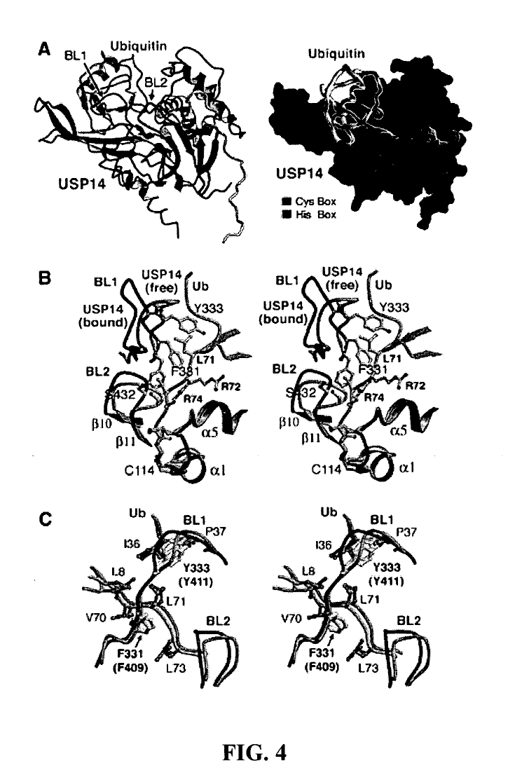

Ubal binds to the predicted ubiquitin-binding surface of USP14 (FIG. 4A). The C terminus of ubiquitin is covalently bonded to the deep catalytic cleft between the Palm and Thumb domains of USP14 via a thiohemiacetal linkage between the Ubal aldehyde group and the side chain of USP14 Cys114. Binding by Ubal induces several prominent conformational changes in the catalytic domain, resulting in an RMSD of 1.3 Å for 325 aligned Cα atoms between the free and Ubal-bound USP14 structures. The recognition between USP14 and Ubal closely resembles that between HAUSP and Ubal.

Conformational Changes in the Active Site Region

Structural comparison between free USP14 and USP14 bound to Ubal revealed that the two surface loops (BL1 and BL2) that hover above the catalytic cleft of free USP14 undergo considerable conformational changes (FIG. 4B). These changes significantly widen the binding groove for the C-terminus of ubiquitin. Structural overlay reveals that the aromatic side chain of Tyr333 in free USP14, which would otherwise sterically clash with Leu71 of the C terminus of ubiquitin in the USP14-Ubal structure, undergoes a 4 Å translation and a 90-degree rotation upon binding to Ubal (FIG. 4B). In addition, Phe331 and Ser 432 in free USP14, which would otherwise clash with Leu73 and Arg74 of ubiquitin, respectively, are translocated over a distance of 3-5 Å. These concerted changes result in the accommodation of the C-terminus of ubiquitin in the newly formed cleft between the Palm and the Thumb domains, thus allowing access of the C-terminal glycyl carbonyl of ubiquitin to the catalytic cysteine Cys114 (FIG. 4B).

The conformational changes of the BL1 and BL2 loops are facilitated by interactions between conserved residues in these loops and the bound ubiquitin moiety. For example, Phe331 in USP14 makes multiple van der Waals contacts to a hydrophobic surface patch formed by Leu8, Val70, Leu71, and Leu73 of the ubiquitin moiety (FIG. 4C). Tyr333 in USP14 also contacts the hydrophobic residues Ile36 and Pro37 of the ubiquitin moiety (FIG. 4C). The interactions mediated by these residues, which block ubiquitin binding in the unliganded USP14, serve to stabilize ubiquitin association. Interestingly, both Phe331 and Tyr333 are conserved in HAUSP (Phe409 and Tyr411) where they make very similar interactions to stabilize substrate binding (FIG. 4C) (Hu et al. (2002) supra). Thus these interactions provide a plausible explanation to the fact that portions of the BL1 sequences are conserved between USP14 and HAUSP (FIG. 2). However, unlike in free USP14, the polypeptide segment corresponding to BL1 does not block the binding cleft for the ubiquitin C-terminus in unliganded HAUSP (Hu et al. (2002) supra). In the Ubal-bound USP14, the BL1 and BL2 loops shift to positions that are comparable to those seen in the structure of the Ubal-bound HAUSP.

Thus, an embodiment of the disclosure provides for a peptide or peptidomimetic, or mimetic of the c-terminal region of ubiquitin. The peptide may be of the sequence:
Val-Leu-X⁺-Leu-X⁺-X-Gly (SEQ ID NO. 1)
   where X⁺ may be a Lys, Arg or Gln, and X may be a Gly, Val, or Ala. A further embodiment of the disclosure provides for a peptide, or mimetic thereof, of the sequence:
X1-X2-X3-X4-X5-X6-X7 (SEQ ID NO. 2)
   where X1 may be Val or a mimetic of Val, X2 may be Leu or a mimetic Leu, X3 may be may be a Lys, Arg or Gln or a mimetic of Lys, Arg or Gln, X4 may be Leu or a mimetic Leu, X5 may be a Lys, Arg or Gln or a mimetic of Lys, Arg or Gln, X6 may be a Gly, Val, or Ala or a mimetic of Gly, Val or Ala, and X7 may be a Gly or a mimetic of Gly. Further, a compound of an embodiment may be a peptide of the sequence:
Val-Leu-Arg-Leu-Arg-Gly-Gly (SEQ ID NO. 3)
   or a mimetic of SEQ ID NO. 3.

Further, another embodiment is directed to a therapeutic composition comprising a peptide of sequence from SEQ ID NOs. 1-3, or a mimetic of a sequence from SEQ ID NOs. 1-3. A method of treating a tumor growth in a patient with cancer comprising administering a therapeutically effective amount of a peptide of sequence from SEQ ID NOs. 1-3, or a mimetic of a sequence from SEQ ID NOs. 1-3, is also disclosed.

A further embodiment of the disclosure provides a compound which is a mimetic of a peptide capable of binding to a catalytic site of a ubiquitin specific processing protease (UBP). The compound may be identified by a method which includes obtaining a set of atomic coordinates defining a three dimensional structure of a crystal of a substrate-UBP complex that effectively diffracts X-rays for the determination of atomic coordinates to a resolution of 5 Angstroms or better. A compound may then be selected that mimics the substrate binding to a catalytic site on the UBP by performing structure based drug design with the atomic coordinates, wherein the selecting may be performed in conjunction with computer modeling. The structure based drug design may be directed compound design or random compound design. This compound may then be contacted with the UBP and binding may be detected. This binding may be done in a cell-free assay, or may be done in a cell-culture assay. The compound may be considered a mimetic if binding of the compound with the catalytic site of the UBP modulates UBP catalytic activity.

In a further embodiment, the crystallographic coordinates for the Ubal-USP14 structure, which are deposited at the protein data bank with accession code 2AYO, may be used as the atomic coordinates which define the three dimensional structure of a crystal of a substrate-UBP complex. The selection which may be performed in conjunction with computer modeling may be selecting a mimetic which is represented by a model that deviates from the atomic coordinates of the substrate by a root mean square deviation of less than 10 angstroms, wherein the substrate may be represented by a peptide which comprises amino acids involved in hydrogen bonding and van der Waals interactions with the catalytic site of UBP. In one embodiment, the mimetic may be a peptide, wherein at least one amino acid may be replaced with a modified amino acid, and at least one bond may be replaced with a peptide bond substitute.

In a further embodiment, the mimetic may be selected using computational screening of one or more databases of chemical compound structures to identify candidate compounds which have structures that are predicted to interact with the catalytic site of the UBP. In yet another embodiment, the mimetic may be capable of inhibiting or activating the activity of USP14. Further, the ubiquitin selective processing protease of the crystal structure may be a mammalian protein, and more preferably, may be USP14.

An embodiment of this disclosure provides a method for testing the ability of a mimetic compound to modulate the deubiquitinating activity of a ubiquitin selective processing protease (UBP). This method may include incubating a mimetic compound with a UBP, adding a tri-ubiquitin tagged at the proximal ubiquitin to the mimetic-UBP mixture, separating components of the above mixture by molecular size, and visualizing the separated components. An identification of a modified level of degradation products of the tri-ubiquitin may demonstrate binding of the mimetic to the UBP. The UBP may be USP14, and the tri-ubiquitin tagged at the proximal ubiquitin may be tagged with a fluorescent label. The fluorescent label may preferably be Lucifer Yellow.

A further embodiment provides for a therapeutic agent or composition which may be a peptide or peptidomimetic, or mimetic of the c-terminal region of ubiquitin. The therapeutic agent may be useful in treating diseases related to USP14, including but not limited to, cancer and neurodegenerative disorders. For example, in some aspects, the disclosure is directed to a pharmaceutical composition comprising a compound, which may be a mimetic of a peptide capable of binding to a catalytic site of a ubiquitin specific processing protease, and a pharmaceutically acceptable carrier or diluent, or an effective amount of a pharmaceutical composition comprising the compound. The disclosure is directed to a pharmaceutical composition comprising a compound, which may be a mimetic of a peptide capable of binding to a surface cleft or binding pocket of a ubiquitin specific processing protease, and a pharmaceutically acceptable carrier or diluent, or an effective amount of a pharmaceutical composition comprising the compound.

Another embodiment is directed to a method of treating a tumor growth in a patient with cancer comprising administering a therapeutically effective amount of a compound which is a mimetic of a peptide capable of binding to a catalytic site or to a surface cleft or binding pocket of a ubiquitin specific processing protease.

Although USP14 and HAUSP share conserved three-domain architecture, they exhibit distinct active site conformations and different activation mechanisms. In free HAUSP, the binding pocket for ubiquitin C-terminus is well formed; but the catalytic triad residues are misaligned and undergo realignment upon binding to ubiquitin. In contrast, the catalytic triad residues are already poised for catalysis in free USP14; however, the binding groove for the C-terminus of ubiquitin is partially filled by two surface loops that undergo significant conformational changes upon binding to ubiquitin. Both mechanisms serve to activate the deubiquitinating activity and appear to ensure appropriate substrate specificity. For USP14, it is possible that association with the 26S proteasome may also facilitate the relief of the steric hindrance posed by the two surface loops, which in turn results in the activation of its deubiquitinating activity

It was recently reported that the Fingers domain of HAUSP resembles a zinc ribbon that has lost its zinc-binding ability (Krishna and Grishin (2004) Cell Cycle 3:1046-1049). This class of zinc ribbon motifs has a characteristic sequence of CX₂CXₙCX₂C, in which the four Cys residues coordinate one zinc atom. In HAUSP, the second, third, and fourth cysteines are replaced by Val, Ala, and His, respectively, and thus zinc is not bound (Hu et al. (2002) supra) (FIG. 2). In USP14 and Ubp6, except for the first Cys in USP14, all other Cys positions of the motif are occupied by amino acids that cannot coordinate the zinc atom (FIG. 2). Thus, although the Fingers domains in these proteins adopt a fold similar to the C4-type zinc ribbon (Krishna and Grishin (2004) supra), they do not bind to zinc. Apparently, zinc binding is not generally required for deubiquitination by UBP-family DUBs. Nonetheless, some UBP proteins, such as yeast Ubp8 and Doa4, contain all four Cys residues and are expected to coordinate a zinc atom.

The structures of the 45-kDa catalytic domain of USP14 in isolation and in a complex with ubiquitin aldehyde reveal two features. First, USP14 indeed contains a three-domain architecture and, like the deubiquitinating enzyme HAUSP, binds to ubiquitin using the Fingers domain and the surface groove between the Palm and the Thumb. Nonetheless, it is important to note that local structural differences exist between HAUSP and USP14.

Second, despite the conservation of this three-domain architecture, the activation mechanism for USP14 appears to be quite different from that for HAUSP. Thus, blocked active sites or misaligned catalytic triads seem to be a common theme for many DUBs, including not just UBP's (Hu et al. (2002) supra; this study) but also UCHs (Johnston et al. (1999) supra; Misaghi et al. (2005) supra). It may be vital to control access to these sites in cells as the catalytic activity of DUBs at inappropriate places or times may lead to unintended deubiquitination. It is worth noting that this conclusion is also supported by the structure of the Ubp6 catalytic core domain (protein data bank accession code 1VJV), which shows a blocked active site by two loops that are very similar to the BL1 and BL2 loops. In addition, our biochemical data show that the Ubl domain of USP14 is responsible for binding to the proteasome and that this binding is required for the activation of the deubiquitinating activity of USP14. Comparison of free and Ubal-complexed USP14 further suggests a mechanism for activation: proteasome-association promotes displacement of two polypeptide loops in USP14 that otherwise block access of substrates to the active site.

Inhibition by BL1 and BL2 may be relieved by the proteasome through a binding interaction of the Ubl domain in USP14/Ubp6 with the proteasome which brings the isopeptidase domain of USP14/Ubp6 close to specific subunit(s) of the proteasome. This may then promote interactions between the BL1/BL2 loops and the proteasome subunits. These interactions in turn relieve the blockade by the BL1/BL2 loops. Alternatively, the binding of the Ubl domain in USP14/Ubp6 by the proteasome may create a novel surface that serves to interact with and change the conformation of the BL1/BL2 loops.

Thus, in an embodiment of the disclosure provides for a peptide or peptidomimetic, or mimetic of a peptide which may bind at the N-terminal of ubiquitin-like region of the USP14. This short peptide or peptidomimetic, or mimetic would mimic the binding of USP14 to the 26S proteasome. A further embodiment of the disclosure provides for a therapeutic agent or composition containing a peptide or peptidomimetic, or mimetic of a peptide which may bind at the N-terminal of ubiquitin-like region of the USP14 which may be useful for the treatment of diseases.

A further embodiment of the disclosure provides a compound which may be a mimetic of a peptide capable of binding to an N-terminal portion of a ubiquitin specific processing protease (UBP). The compound may be identified by a method which may include obtaining a set of atomic coordinates defining a three dimensional structure of a crystal of a UBP that effectively diffracts X-rays for the determination of atomic coordinates to a resolution of 5 Angstroms or better. A compound may then by selected that binds to a ubiquitin-like N-terminus of the UBP by performing structure based drug design with the atomic coordinates, wherein the selecting may be performed in conjunction with computer modeling. The structure based drug design may be directed compound design or random compound design. This compound may then be contacted with the UBP and binding may be detected. This binding may be done in a cell-free assay, or may be done in a cell-culture assay. The compound may be considered a mimetic if binding of the compound with UBP modulates UBP catalytic activity.

In a further embodiment, the crystallographic coordinates for the USP14 structure, which are deposited at the protein data bank with accession code 1 AYN, may be used as the atomic coordinates which define the three dimensional structure of a crystal of a UBP. In one embodiment, the mimetic may be a peptide, wherein at least one amino acid may be replaced with a modified amino acid, and at least one bond may be replaced with a peptide bond substitute.

In a further embodiment, the mimetic may be selected using computational screening of one or more databases of chemical compound structures to identify candidate compounds which have structures that are predicted to interact with the N-terminal portion of a ubiquitin specific processing protease. In yet another embodiment, the mimetic may be capable of inhibiting or activating the activity of USP14. Further, the ubiquitin selective processing protease of the crystal structure may be a mammalian protein, and more preferably, may be USP14.

At present, structural information is available on two UBP proteins (HAUSP and USP14) and their complexes with Ubal, which reveal two quite different activation mechanisms. In the case of HAUSP, the active site conformation is realigned through substrate binding; in the case of USP14, two surface loops (BL1 and BL2) are displaced to widen the binding groove for the ubiquitin C-terminus.

Continued biochemical and structural investigation may reveal additional novel mechanisms for the activation of the active sites in UBP's. For example, ubiquitin binding by the Fingers domain may be a regulated event, and the concave surface of the Fingers domain may be occupied by the N- or C-terminal polypeptide that extends from the isopeptidase domain of an UBP. Alternatively, these N- or C-terminal extensions may directly interact with the isopeptidase domain to deform its active site or ubiquitin-binding site. Structural investigation of the UBP's and their cognate complexes with substrate may reveal additional insights into their functions and mechanisms.

Thus, one embodiment of the disclosure is a method for identifying an inhibitor of USP14 activity as a deubiquitinating protease. In the method, a three dimensional structure derived by X-ray diffraction from a crystal of USP14 or USP14-Ubal is provided and then employed to design or select a potential inhibitor. The potential inhibitor is synthesized and a determination may be made as to whether or not it can inhibit the protease activity of USP14. The inhibitor may be a peptide which binds reversibly or irreversibly to the active site of USP14. Further, the inhibitor may be a peptide, peptidomimetic or mimetic thereof which binds to the BL1 or BL2 loops and does not allow the conformational changes which open or expose the active site.

In another embodiment of the disclosure, a method for identifying an activator of USP14 activity as a deubiquitinating protease is provided. In the method, a three dimensional structure derived by X-ray diffraction from a crystal of USP14-Ubal is provided and then employed to design or select a potential activator, based on the conformational change at the active site of USP14 associated with the Ubal binding. The potential activator is synthesized and a determination may be made as to whether or not it can activate the protease activity of USP 14.

In another embodiment of the instant disclosure, the USP14 binding peptides, which are antagonists or agonists, are modified to produce peptide mimetics by replacement of one or more naturally occurring side chains of the 20 genetically encoded amino acids (or D amino acids) with other side chains. For example, the other side chains may contain groups such as alkyl, lower alkyl, cyclic 4-, 5-, 6-, to 7-membered alkyl, amide, amide lower alkyl, amide di(lower alkyl), lower alkoxy, hydroxyl, carboxy and the lower ester derivatives thereof, and with 4-, 5-, 6-, to 7-membered heterocyclics. For example, praline analogs can be made in which the ring size of the proline residue is changed from 5 members to 4, 6 or 7 members. Cyclic groups can be saturated or unsaturated, ad if unsaturated, can be aromatic or non-aromatic. Heterocyclic groups can contain one or more nitrogen, oxygen, and/or sulfur heteroatoms. Examples of such groups include furazanyl, furyl, imidazolidinyl, imidazolyl, imidazolinyl, isothiazolyl, isoazolyl, morpholinyl (e.g. morpholino), oxazolyl, piperazinyl (e.g. 1-piperazinyl), piperidyl (e.g. 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g. 1-pyrrolidinyl), pyrrolinyl, pyrrolyl, thiadiazolyl, thiazolyl, thienyl, thiomorpholinyl, (e.g. thiomorpholino), and triazolyl. These heterocyclic groups can be substituted or unsubstituted. Where a group is substituted, the substituent can be alkyl, alkoxy, halogen, oxygen, or substituted or unsubstituted phenyl. Peptidometics may also have amino acid residues that have been chemically modified by phosphorylation, sulfonation, biotinylation, or the addition or removal of other moieties.

A variety of techniques are available for constructing peptide mimetics with the same or similar desired biological activity as the corresponding native, but with more favorable activity than the peptide with respect to solubility, stability, and/or susceptibility to hydrolysis or proteolysis (Morgan et al. (1989) Ann Rep Med Chem 24:243-252). Certain peptidomimetic compounds are based upon the amino acid sequence of the peptides of the disclosure. Often, peptidomimetic compounds are synthetic compounds having a three dimensional structure (i.e. a “peptide” motif) based upon the three dimensional structure of a selected peptide. The peptide motif provides the peptidomimetic compound with the desired biological activity, i.e. binding to USP14 or other members of the UBP family, wherein the binding activity of the mimetic compound is not substantially reduced, and is often the same as or greater than the activity of the native peptide on which the mimetic was modeled. Peptidomimetic compounds can have additional characteristics that enhance their therapeutic application, such as increased cell permeability, greater affinity and/or avidity and prolonged biological half-life.

Peptidomimetic design strategies are available in the art (Ripka et al. (1998) Curr Opin Chem Biol 2:441-452; Hruby et al. (1997) Curr Opin Chem Biol 1:114-119; Hruby et al. (2000) Curr Med Chem 9:945-970). One class of peptidomimetic mimics a backbone that is partially or completely non-peptide, but mimics the peptide backbone atom-for-atom and comprises side groups that likewise mimic the functionality of the side groups of the native amino acid residues. Several types of chemical bonds e.g. ester, thioester, thioamide, retroamide, reduced carbonyl, dimethylene and ketomethylene bonds, are known in the art to be generally useful substitues for peptide bonds in the construction or protease resistant peptidomimetics. Another class of peptidomimetics comprises a small non-peptide molecule that binds to another peptide or protein, but which is not necessarily a structural mimetic of the native peptide.

Yet another class of peptidomimetics has arisen from combinatorial chemistry and the generation of massive chemical libraries. These generally comprise novel templates which, though structurally unrelated to the native peptide, possess necessary functional groups positioned on a non-peptide scaffold to serve as “topographical” mimetics of the original peptide (Ripka et al. (1998) supra).

In another embodiment, the USP14 binding peptides or peptidomimetics are used in a cell free binding assay to screen for USP14-binding antagonists and agonists. Such assays are well known to persons of skill in the art, and are particularly useful for high throughput screening for candidate drugs, e.g. in a chemical library or produced by rational drug design (mimetics) as disclosed herein.

In another embodiment of the disclosure, the USP14 binding peptides or peptidomimetics are utilized in various assays to screen for and identify compounds capable of acting as antagonists or agonists of the USP14 catalytic activity. Antagonists of this activity are expected to be useful as anti-cancer drugs for the treatment of the aberrant cell proliferation observed in cancer. Agonists of this activity are expected to be useful in regulating synaptic activity in mammals for the treatment of neurodegenerative diseases.

In yet another embodiment of the instant disclosure, these small molecules or peptidomimetics or mimetics which are antagonists or agonists of USP14 activity may be used as a therapeutic for the treatment of diseases such as cancer or neurodegenerative diseases. Accordingly, an embodiment of the disclosure comprises administering to a cell a therapeutically effective amount of the compounds to stimulate or inhibit the activity of USP14. Stimulating the USP14 activity may be useful for the treatment of neurodegenerative diseases, while inhibiting the activity may be useful for the treatment of cancer. In another embodiment, the cell is contained within a tissue, and the tissue preferably is located in a living organism, preferably an animal, more preferably a mammal, and most preferably a human.

These later embodiments of the disclosure are carried out by formulating the mimetics described herein as pharmaceutical preparations or therapeutic compositions for administration in a subject. Such a pharmaceutical preparation constitutes another aspect of the disclosure. For example, in some aspects, the disclosure is directed to a pharmaceutical composition comprising a compound, as defined above, and a pharmaceutically acceptable carrier or diluent, or an effective amount of a pharmaceutical composition comprising a compound as defined above.

The compounds of the present invention can be administered in the conventional manner by any route where they are active. Administration can be systemic, topical, or oral. For example, administration can be, but is not limited to, parenteral, subcutaneous, intravenous, intramuscular, intraperitoneal, transdermal, oral, buccal, or ocular routes, or intravaginally, by inhalation, by depot injections, or by implants. Thus, modes of administration for the compounds of the present invention (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Specific modes of administration will depend on the indication. The selection of the specific route of administration and the dose regimen is to be adjusted or titrated by the clinician according to methods known to the clinician in order to obtain the optimal clinical response. The amount of compound to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician).

Pharmaceutical formulations containing the compounds of the present invention and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of the present invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The compounds of the present invention can be formulated for parenteral administration by injection, e.g., by bolus injection or continuous infusion. The compounds can be administered by continuous infusion subcutaneously over a period of about 15 minutes to about 24 hours. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the compounds can be formulated readily by combining these compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, e.g., tablets or lozenges formulated in a conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compounds of the present invention can also be formulated in rectal compositions such as suppositories or retention enemas, e.g., containing conventional suppository bases such as cocoa butter or other glycerides.

In addition to the formulations described previously, the compounds of the present invention can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In transdermal administration, the compounds of the present invention, for example, can be applied to a plaster, or can be applied by transdermal, therapeutic systems that are consequently supplied to the organism.

Pharmaceutical compositions of the compounds also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The compounds of the present invention can also be administered in combination with other active ingredients, such as, for example, adjuvants, protease inhibitors, or other compatible drugs or compounds where such combination is seen to be desirable or advantageous in achieving the desired effects of the methods described herein.

This invention and embodiments illustrating the method and materials used may be further understood by reference to the following non-limiting examples.

### EXAMPLES

### Example 1

### Generation of Constructs and Protein Purification

All constructs used or listed in this disclosure were generated using a standard PCR-based cloning strategy. For crystallization purposes, the catalytic core domain of USP14 (91-494) was cloned into the pET-15b vector (Pharmacia), and was overexpressed in Escherichia coli strain BL21 (DE3) as an N-terminally His6-tagged protein. Seleno-Met-substituted USP14 (91-494) was expressed in Escherichia coli B834 (DE3) (Novagen) in M9 minimal medium supplemented with 50 mg/l selenomethionine. For in vitro deubiquitination assay using Ly-Ub₃, the full-length USP14 was cloned into pET-15b (Pharmacia), overexpressed in BL21 (DE3) as an N-terminally His₆-tagged protein. For proteasome association and activation assays, the full-length USP14 and USP14 (91-494) were cloned into the vector pGEX-2T (Pharmacia), and the USP14 ubiquitin-like domain (UBL; residues 1-90) was cloned into the vector pGEX-4T-1 (Pharmacia). Protein purification followed the general procedure described (Hu et al. (2002) supra).

Ubal samples, as disclosed herein, were prepared by carboxypeptidase Y-catalyzed exchange of 3-amino-1,2-propanediol for ubiquitin Gly76 and the subsequent oxidation of the ubiquitin-diol product with NaIO₄. The Ubal thus obtained was incubated in 4-fold excess over USP14 (91-494) protein at pH 8 (25 mM Tris, 100 mM NaCl, 5 mM DTT), and the USP14-Ubal complex was isolated by gel filtration (Superdex 200, 10 mM Tris, pH 8.0, 100 mM NaCl, 4 mM DTT).

### Example 2

### Crystallization, Data Collection and Structure Determination for Free USP14

Crystals of this disclosure were grown by the hanging-drop method by mixing the USP14 protein (residues 91-494) (˜15 mg/ml) with an equal volume of reservoir solution containing 100 mM MES pH 6.5, 200 mM (NH₄)2SO₄, and 30% PEGMME 5000 (w/v). Small crystals appeared overnight and were used as seeds to generate larger crystals from Seleno-Met USP14 protein. The crystals belong to the spacegroup P212121, with a=82.29 Å, b 121.58 Å, c=166.85 Å. Crystals were equilibrated in a cryoprotectant buffer containing reservoir buffer plus 20% glycerol (v/v) and were flash frozen in a cold nitrogen stream at −170° C. The native and MAD date sets were collected at NSLS beamline X-25 and CHESS F-2, respectively, and were processed using the software Denzo and Scalepack (Otwinowski and Minor (1997) Methods Enzymol 276:307-326).

Eighteen out of the 39 selenium sites were determined using SOLVE (Terwilliger and Berendzen (1996) Acta Crystallogr D52:749-757) and the initial experimental phases were calculated and improved by solvent flattening using DM (Collaborative Computational Project (1994) Acta Crystallogr D50:760-763). The electron density map allowed the manual identification and docking of three copies of HAUSP catalytic domain (Hu et al. (2002) supra), a homolog of USP14. After rigid body refinement, the non-crystallographic symmetry between the three molecules allowed the identification of 30 selenium atoms from the 18 sites determined by SOLVE. The atomic model was built using O(Jones et at. (1991) Acta Crystallogr A47:110-119) and refined using CNS (Brunger et al. (1998) Acta Crystallogr D54:905-921). The heavy atom parameters were refined and new experimental phases were calculated with CNS. Three more selenium sites were determined by anomalous difference Fourier synthesis using diffraction data at the peak wavelength. After completing the heavy atom model, the new experimental phases were extensively improved by three-fold NCS averaging and extended to 3.2 Å in DM. The electron density map after DM was clear and continuous in most parts of the molecules. The final model contains three molecules in each asymmetric unit, each containing amino acids 99-216, 235-379, and 398-483. The average B factor is 123.4 Å² for all atoms and the estimated coordinate error is 0.54 Å. No residue is in the disallowed region of the Ramachandran Plot. There is no significant electron density for residues 94-98, 217-234, 380-397, and 484-494; these residues are likely flexible and disordered in the crystals.

### Example 3

### Crystallization and Structure Determination of the USP14-Ubal Complex

Crystals were grown by the hanging-drop method by mixing the complex (˜10 mg/ml) with an equal volume of reservoir solution containing 100 mM Tris pH 8.0, 100 mM CaCl₂, 25% PEG1000. The crystals belong to the space group P3121, with a=b=183.9 Å and c=45.7 Å. The native date set was collected at CHESS A-1, and processed using the software Denzo and Scalepack (Otwinowski and Minor (1997) supra). The structure was determined by molecular replacement using AMoRe (Navaza (1994) Acta Crystallogr A50:157-163) and refined using CNS (Terwilliger and Berendzen (1996) supra). The average B factor is 93.4 Å² for all atoms and the estimated coordinate error is 0.81 Å. No residue is in the disallowed region of the Ramachandran Plot.

### Example 4

### In Vitro Deubiquitination Assays

Most protein substrates targeted for proteasome degradation are conjugated to polyubiquitin chains (Pickart and Cohen (2004) supra; Thrower et al. (2000) Embo J 19:94-102). USP14 is thought to play an important role in removing the ubiquitin moiety from polyubiquitinated substrates. However, the substrate specificity for USP14 remains unclear. Among the several unanswered questions, it is not known whether USP14 prefers to cleave the proximal ubiquitin from the polyubiquitin chain, or whether it instead progressively shortens polyubiquitin chains from the distal end.

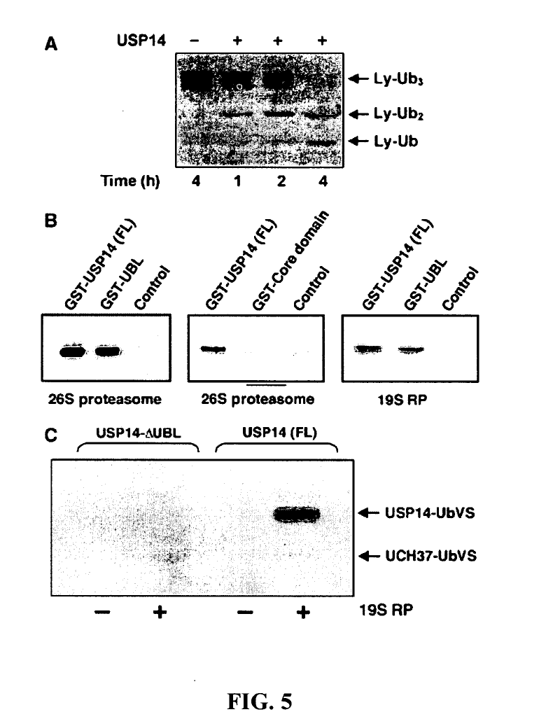

To examine this aspect of substrate specificity of USP14 for polyubiquitin disassembly, we reconstituted an in vitro deubiquitination assay using Lys48-linked Ub₃ as a model substrate, and monitored temporal appearance of cleavage products (FIG. 5A). In this triUb substrate, ubiquitin at the proximal end is fluorescently labeled by Lucifer Yellow. In the initial stage of reaction, the major deubiquitination products were found to be fluorescently labeled di-ubiquitin (FIG. 5A) and unlabeled mono-ubiquitin (invisible at the one hour time point in FIG. 5A). As the reaction proceeded to completion, the fluorescently labeled di-ubiquitin was further reduced to mono-ubiquitin. Although the data shown here (FIG. 5A) were obtained using the full-length USP14, similar results were obtained for the Ubl-deleted USP14 (residues 91-494). This observation indicates that USP14 prefers to cleave ubiquitin from the distal end of a Lys48-linked polyubiquitin chain. This conclusion was also confirmed using Lys48-linked Ub4 and Ub5 as model substrates. We also tested whether USP14 can cleave alternatively linked ubiquitin oligomers such as Lys63-linked di-ubiquitin. The cleavage was extremely slow and was almost at the detection limit; hence we concluded that Lys63-linked ubiquitin oligomers were unlikely to be substrates for USP14.

K48-linked tri-ubiquitin labeled with Lucifer Yellow at the proximal ubiquitin (Ly-Ub₃) was made from Ub-Ub-Ub (T66C) as previously described (Lam et al. (1997) supra). To examine the substrate specificity of USP14, recombinant full-length USP14 (6 nM, or none in a control reaction) was incubated with 2 μM Ly-Ub₃ in the reaction buffer containing 50 mM HEPES pH 8.0, 50 mM NaCl, 1 mM EDTA, 5 mM DTT, and 0.1 mg/ml ovalbumin, at 37° C. for the indicated time. The disassembly of fluorescent Ly-Ub₃ was visualized with a cooled CCD camera system (BioChemi System, UVP Biolmaging) after separation by SDS-PAGE. Products of incubations with K63-linked polyubiquitin chains (a gift from C. Pickart, Johns Hopkins University, Baltimore) were evaluated by SDS-PAGE followed by silver-staining or detection after transfer to nitrocellulose with mouse monoclonal anti-ubiquitin antibody (clone P4D1; Santa Cruz).

### Example 5

### In Vitro Proteasome Association Assay

USP14 was previously reported to associate with the 26S proteasome (Borodovsky et al. (2001) supra), but the exact mechanism of recognition was not elucidated. To further characterize this interaction between USP14 and the proteasome, we purified fusion proteins between glutathione S-transferase (GST) and the full-length USP14 protein, the Ubl domain, or the catalytic domain. Then we examined their interaction with the proteasome in a GST-mediated pull-down assay. After extensive wash, the bound proteasome was eluted using 10 mM reduced glutathione and detected by an antibody specific for the proteasomal S1 subunit. Both full-length USP14 and the Ubl domain bound efficiently to the 26S proteasome (FIG. 5B). In contrast, no significant binding was detected between the catalytic domain of USP14 and the proteasome. The USP14 binding site in the proteasome was further mapped to the 19S regulatory particle (RP) of the proteasome, as both full-length USP14 and the Ubi domain specifically bound to the 19S RP (FIG. 5B) but not the 20S proteasome catalytic core particle. The binding efficiency of full-length USP14 to the 19S RP appears to be higher than that of the Ubl domain, suggesting that additional interaction may involve the catalytic domain. These findings are consistent with the reported observations that, in yeast, the full-length Ubp6 associates with proteasomes more efficiently than the Ubi domain alone (Chernova et al. (2003) supra; Leggett et al. (2002) supra).

26S proteasome purified from bovine erythrocytes was available from previous experimentation (Yao and Cohen (2002) supra). The bovine 19S complex (PA700) was a gift from G. DeMartino (UT Southwestern Medical Center, Dallas). An approximately equi-molar amount (0.35 nmole) of GST-USP14, GST-USP14 (91-494), GST-UBL, or GST (control) was mixed with either 2.7 pmole 26S proteasome or 1.57 pmole 19S complex (PA700) in the binding assay buffer containing 50 mM Tris, pH 7.5, 1 mM ATP, 1 mM MgCl₂, 10 mM DTT, 0.1 mg/ml ovalbumin, and 10% glycerol. The mixture was incubated with glutathione Sepharose resin at 4° C. for 1 hour, followed by extensive wash using the binding assay buffer. Proteins bound to the resin were eluted with 10 mM reduced glutathione in 50 mM Tris, pH 8.0. Eluted proteins were resolved by 10% SDS-PAGE, transferred to nitrocellulose membrane, and probed with mouse monoclonal anti-Si subunit antibody (Affiniti) as a marker for the 19S or 26S proteasome complex. Membrane blots were probed with anti-GST antibody to assure comparable loadings of resin with GST and GST-fusion proteins.

### Example 6

### Activation of USP14 by Proteasome Association

Ubiquitin vinylsulfone (UbVS) is a specific covalent inhibitor of a large subset of deubiquitinating enzymes, and thus can be used as an active site-directed probe for the detection of DUB activity (Borodovsky et al. (2001) supra). In order to examine the effect of proteasome association on the activity of USP14, the purified recombinant USP14 proteins were pre-incubated with or without the 19S RP and then assayed for covalent modification by UbVS.

The USP14-ΔUBL protein, which lacks the ability to bind to proteasome, showed extremely low activity toward UbYS, both in the absence and presence of the 19S regulatory particle (FIG. 5C, lanes 1 and 2). The full-length USP14 by itself also exhibited extremely low activity toward UbVS, as there was no detectable USP14-UbVS complex formation (FIG. 5C, lane 3). However, addition of the 19S regulatory particle to the full-length USP14 sample greatly increased its activity toward UbVS, as indicated by the appearance of a strong USP14-UbVS band (FIG. 5C, lane 4).

Relative to the USP14 adduct, only a trace amount of UCH37-UbVS was detected in reactions in the presence of 19S RP (FIG. 5C, lanes 2 and 4). Two factors contribute to this observation. First, UCH37 appears to be intrinsically less reactive with (UbVS than is the activated form of USP14; notably, this is despite the higher activity shown by UCH37 with the substrate ubiquitin-AMC. Second, whereas UCH37 was present in the reactions as a stoichiometric component of the 19S RP complex (Lam et al. (1997) supra), USP14 was in a 22-fold molar excess. The results suggest that USP 14 (and the USP 14-UbVS adduct) can exchange between the populations of free and proteasome-bound enzyme.

Six ATPases are prominent among the subunits of the 19S RP (Pickart and Cohen (2004) supra), and therefore we tested whether USP14 activation requires ATP binding or hydrolysis. No differences in reactivity with UbVS were observed when reactions that contained 1 mM ATP were compared with those in which ATP was depleted by apyrase (data not shown). The state of the proteasome catalytic chamber may also influence USP14 activity. This possibility was suggested by the observation that treatment of cultured mammalian cells with any of several irreversible proteasome inhibitors facilitated labeling of USP14 by UbVS in cell extracts (Borodovsky et al. (2001) supra). However, when we compared USP14 activation in vitro by 26S proteasomes preincubated with either zero (control) or 4 μM epoxomycin, both samples showed equal reactivity with UbVS; assays with the fluorogenic peptide substrate N-succinyl-LLVY-7-amino-4-methylcoumarin confirmed that the proteasomes were inhibited completely by the epoxomycin (data not shown). Thus, the reported effect of proteasome inhibitors in vivo on USP14 activation is most likely indirect and might have been due to increased amounts of either total or proteasome-associated USP14.

The results above show that, as assessed by reaction with UbVS, USP14 activation is a consequence of binding to the 19S RP and is independent of the ATPase activities and the 20S proteolytic core of the proteasome. Thus, the simplest mechanism to explain USP14 activation is that interactions with subunit(s) in the 19S RP complex promote movement of loops BL1 and BL2 to make the active site cleft accessible to ubiquitin. This mechanism shares features with DUBs of the UCH family which similarly have a loop that occludes the active site (Johnston et al. (1999) supra; Misaghi et al. (2005) J Biol Chem 280:1512-1520). However, the UCHs are fundamentally different in that a single loop blocks the active site by crossing over it rather than flanking and narrowing the cleft.

Moreover, the active site crossover loop residues in the UCH enzymes appear to control activity by interfering with binding of the ubiquitin-conjugated protein or peptide rather than the ubiquitin moiety itself (Johnston et al. (1999) supra; Misaghi et al. (2005) supra). Whereas loop conformation may switch USP14 between ubiquitin binding and nonbinding states (active and inactive states, respectively), the active site crossover loop in UCH enzymes instead may act as a filter that can discriminate among different ubiquitin conjugates.

UbVS was prepared as previously described (Borodovsky et al. (2001) supra). To test the activation of USP14 upon proteasome binding, 3.5 μM full-length USP14 or USP14-AUBL (91-494) was incubated with or without 157 nM bovine 19S complex (PA700) in the binding buffer containing 50 mM Tris pH 7.5, 1 mM ATP, 1 mM MgCl₂, 10 mM DTT, 0.1 mg/ml ovalbumin, and 10% glycerol, at 4° C. for 1 hour. UbVS was then added to the sample to give approximately 1:1 stoichiometry with the 19S complex. Incubation was continued for another hour and then stopped by the addition of 2×SDS sample buffer. The reaction mixtures were resolved by 14% SDS-PAGE, transferred to nitrocellulose membrane, and the UbVS adducts were detected with mouse monoclonal anti-ubiquitin antibody (P4D1) (Santa Cruz).

Although the present invention has been described in considerable detail with reference to certain preferred embodiments thereof, other versions are possible. Therefore the spirit and scope of the appended claims should not be limited to the description and the preferred versions contained within this specification.

What is claimed is:

1. A compound comprising a mimetic of a peptide capable of binding to a surface cleft of a ubiquitin specific processing protease (UBP) wherein the mimetic has a three dimensional structure complementary to the surface cleft of the UBP defined by atomic coordinates of a substrate-UBP complex.

2. The compound of claim 1, wherein the atomic coordinates defining the three dimensional structure of the substrate-UBP complex are those with protein data bank accession code 2AYO.

3. The compound of claim 1, wherein the mimetic has a three dimensional structure which is represented by a model that deviates from the atomic coordinates of the c-terminus of the substrate by a root mean square deviation of less than 10 angstroms.

4. The compound of claim 3, wherein the c-terminus of the substrate is represented by a peptide which comprises amino acids involved in hydrogen bonding and van der Waals interactions with the residues in the surface cleft of UBP.

5. The compound of claim 1, wherein the substrate is ubiquitin.

6. The compound of claim 1, wherein the mimetic is a peptide.

7. The compound of claim 6, wherein at least one amino acid of the peptide is replaced with a modified amino acid.

8. The compound of claim 6, wherein at least one bond in the peptide is replaced with a peptide bond substitute.

9. The compound of claim 1, wherein the mimetic is capable of inhibiting the activity of USP14.

10. The compound of claim 1, wherein the ubiquitin selective processing protease is USP14.

11. A peptide capable of binding to a surface cleft of a ubiquitin specific processing protease (UBP).

12. The peptide of claim 11, wherein the peptide is Val-Leu-X1-Leu-X1-X2-Gly, wherein X1 is Lys, Arg or Gln, and X2 is Gly, Val, or Ala.

13. The peptide of claim 11, wherein the peptide is Val-Leu-Arg-Leu-Arg-Gly-Gly.

14. The peptide of claim 11, wherein the peptide is capable of modulating UBP catalytic activity.

15. The peptide of claim 11, wherein the ubiquitin specific processing protease is USP14.

16. A synthetic peptide having the sequence X1-X2-X3-X4-X5-X6-X7, wherein
X1 is Val or a mimetic of Val;
X2 is Leu or a mimetic of Leu;
X3 is Arg, Lys or Gln, or a mimetic of Arg, Lys or Gln;
X4 is Leu or a mimetic of Leu;
X5 is Arg, Lys or Gln, or a mimetic of Arg, Lys or Gln;
X6 is Gly, Val or Ala, or a mimetic of Gly, Val or Ala; and
X7 is Gly or a mimetic of Gly.

17. The synthetic peptide of claim 16, wherein X1-X2-X3-X4-X5-X6-X7 is Val-Leu-Arg-Leu-Arg-Gly-Gly.

18. The synthetic peptide of claim 16, wherein at least one bond in the peptide is replaced with a peptide bond substitute.

19. The synthetic peptide of claim 16, wherein the peptide binds to and modulates catalytic activity of a ubiquitin specific processing protease.

20. The synthetic peptide of claim 16, wherein the ubiquitin specific processing protease is USP14.

21. A method of identifying a compound that modulates catalytic activity of ubiquitin specific processing proteases (UBP), comprising:
obtaining a set of atomic coordinates defining a three dimensional structure of a crystal of a substrate-UBP complex that effectively diffracts X-rays for the determination of atomic coordinates to a resolution of 5 Angstroms or better;
selecting a compound that mimics the substrate binding to the catalytic site on the UBP by performing structure based drug design with the atomic coordinates obtained, wherein said selecting is performed in conjunction with computer modeling;
contacting the compound with the UBP; and
detecting binding of the compound with the catalytic site of the UBP, wherein the compound is selected if it is capable of modulating UBP catalytic activity.

22. The method of claim 21, wherein the atomic coordinates defining the three dimensional structure of the substrate-UBP complex are those with protein data bank accession code 2AYO.

23. The method of claim 21, wherein selecting performed in conjunction with computer modeling is selecting a mimetic which is represented by a model that deviates from the atomic coordinates of the substrate by a root mean square deviation of less than 10 angstroms, wherein the substrate is represented by a peptide which comprises amino acids involved in hydrogen bonding and van der Waals interactions with the catalytic site of UBP.

24. The method of claim 21, wherein performing structure based drug design may comprise computational screening of one or more databases of chemical compound structures to identify candidate compounds which have structures that are predicted to interact with the catalytic site of the UBP.

25. The method of claim 21, wherein the ubiquitin selective processing protease is USP14.

26. The method of claim 21, wherein the substrate-UBP complex is an ubiquitin aldehyde-UBP complex.


SEQUENCE LISTING (3 sequences)

SEQ ID NO: 1 - protein, 7 aa, Artificial
  C-terminal 7 amino acids of ubiquitin
  misc_feature (3)..(3): Xaa can be Lys, Arg or Gln
  misc_feature (5)..(5): Xaa can be Lys, Arg or Gln
  misc_feature (6)..(6): Xaa can be Gly, Val or Ala
VLXLXXG

SEQ ID NO: 2 - protein, 7 aa, mammalian
  misc_feature (1)..(1): Xaa can be Val or a mimetic of Val
  misc_feature (2)..(2): Xaa can be Leu or a mimetic of Leu
  misc_feature (3)..(3): Xaa can be Lys, Arg or Gln or a mimetic of Lys, Arg or Gln
  misc_feature (4)..(4): Xaa can be Leu or a mimetic of Leu
  misc_feature (5)..(5): Xaa can be Lys, Arg or Gln or a mimetic of Lys, Arg or Gln
  misc_feature (6)..(6): Xaa can be Gly, Val or Ala or a mimetic of Gly, Val or Ala
  misc_feature (7)..(7): Xaa can be Gly or a mimetic of Gly
XXXXXXX

SEQ ID NO: 3 - protein, 7 aa, mammalian
VLRLRGG